(19) United States
(12) Patent Application Publication
(10) Pub. No.: US 2013/0018426 A1
(43) Pub. Date: Jan. 17, 2013

(54) Bone Plate Module with Adjustable Fixing Direction and an Adjustment Ring Thereof

(71) Applicants: Feng-Te Tsai, Kaohsiung City (TW); Chih-Kun Hsiao, Kaohsiung City (TW); Ting-Sheng Lin, Kaohsiung City (TW); Yuan-Kun Tu, Kaohsiung City (TW); Ching-Hou Ma, Kaohsiung City (TW); Chin-Hsien Wu, Kaohsiung City (TW)

(72) Inventors: Feng-Te Tsai, Kaohsiung City (TW); Chih-Kun Hsiao, Kaohsiung City (TW); Ting-Sheng Lin, Kaohsiung City (TW); Yuan-Kun Tu, Kaohsiung City (TW); Ching-Hou Ma, Kaohsiung City (TW); Chin-Hsien Wu, Kaohsiung City (TW)

(21) Appl. No.: 13/179,618

(22) Filed: Jul. 11, 2011

(51) Int. Cl. A61B 17/80

(52) U.S. Cl. 606/290

(57) ABSTRACT

A bone plate module with adjustable fixing direction includes a bone plate, an adjustment ring and a bone nail. The bone plate has a coupling hole. The adjustment ring is disposed in the coupling hole and has a thread portion. The adjustment ring further comprises a first adjustment member and a second adjustment member. The first adjustment member has a first end coupled with a first end of the second adjustment member. The bone nail has an adjustment portion coupled with the thread portion of the adjustment ring.

### BACKGROUND OF THE INVENTION

1. Field of the Invention

The present invention generally relates to a bone plate module and an adjustment ring thereof and, more particularly, to a bone plate module with adjustable fixing direction that provides a desired positioning effect on an injured part with fracture, as well as an adjustment ring thereof.

2. Description of the Related Art

Referring to FIG. 1, Taiwanese Patent No. M374840 discloses a conventional bone plate module 9 with adjustable fixing direction, which includes a bone plate 91, a screwing ball 92 and a bone nail 93. The bone plate 91 has a plurality of holes 911, each having a spherical wall. The screwing ball 92 has a thread hole 921 at a center thereof. The screwing ball 92 also includes a gap 922 extending from a circumferential wall of the thread hole 921 to an outer spherical face of the screwing ball 92. During assembly, the screwing ball 92 should be stuffed into one of the holes 911. In addition, the bone nail 93 has a thread 931 which allows the bone nail 93 to be screwed through the thread hole 921 of the screwing ball 92.

When the bone plate module 9 is in use, the bone plate 91 is placed on an injured part of a patient where fracture takes place. Then, the screwing ball 92 is stuffed into a desired hole 911 so that the bone nail 93 can be screwed through the thread hole 921 of the screwing ball 92. When the bone nail 93 is being screwed through the thread hole 921, the bone nail 93 will hold open the gap 922 of the screwing ball 92, forcing the outer spherical surface of the screwing ball 92 to touch and abut against the spherical wall of the hole 911. Thus, the bone plate 91 can be well positioned on the injured part.

In the above structure, the bone plate module 9 can keep the screwing ball 92 in the hole 911 without disengagement based on the radial expansion of the screwing ball 92 caused by the bone nail 93 driven through the thread hole 921 of the screwing ball 92. However, the radial expansion range is somewhat limited because only a single gap 922 is arranged. Specifically, the single gap 922 alone cannot cause sufficient radial expansion of the screwing ball 92, thus leading to a poor abutting between the outer spherical surface of the screwing ball 92 and the spherical wall of the hole 911. This deteriorates the coupling effect between the bone plate 91 and the screwing ball 92 and therefore causes loosening of the bone plate module 9 upon the injured part. As a result, healing of the injured part is affected. Another concern is that the screwing ball 92 tends to rotate in the hole 911 before the bone nail 93 is driven through the thread hole 921 of the screwing ball 92. Therefore, it will be troublesome to align the bone nail 93 with the thread hole 921. In light of the above problems, it is desired to improve the conventional bone plate module 9.

### SUMMARY OF THE INVENTION

It is therefore the primary objective of this invention to provide a bone plate module with adjustable fixing direction that offers a desired positioning effect on an injured part with fracture for better healing effect of the injured part.

It is another objective of this invention to provide an adjustment ring that can be disposed in a bone plate in a stable way.

The invention discloses a bone plate module with adjustable fixing direction, which includes a bone plate, an adjustment ring and a bone nail. The bone plate has a coupling hole. The adjustment ring is disposed in the coupling hole and has a thread portion. The adjustment ring further comprises a first adjustment member and a second adjustment member. The first adjustment member has a first end coupled with a first end of the second adjustment member. The bone nail has an adjustment portion coupled with the thread portion of the adjustment ring.

Furthermore, the invention discloses an adjustment ring of a bone plate module with adjustable fixing direction, which includes a first adjustment member and a second adjustment member. The first adjustment member has a first end and a first thread portion. The second adjustment member has a first end and a second thread portion. The first ends of the first and second adjustment members are connected to each other, and the first and second thread portions jointly form a thread portion.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 is an exploded view of a conventional bone plate module.

FIG. 2 is an exploded view of a bone plate module with adjustable fixing direction according to a preferred embodiment of the invention.

FIG. 3 is a cross-sectional view of the bone plate module of the invention.

FIG. 4 is a top view of the bone plate module of the invention observed at line 4-4 in FIG. 3.

FIG. 5 is another cross-sectional view of the bone plate module of the invention showing an exemplary use of the bone plate module where an adjustment ring thereof is disposed in a certain inclination.

FIG. 6 is yet another cross-sectional view of the bone plate module of the invention showing another exemplary use of the bone plate module where the adjustment ring disposed in the inclination is broken into two pieces.

FIG. 7 is a top view of the bone plate module of the invention observed at line 7-7 in FIG. 6.

FIG. 8 is an application of the bone plate module of the invention where the bone plate module is placed on an injured part with fracture.

FIG. 9 is a top view of the bone plate module of the invention according to another implementation thereof.

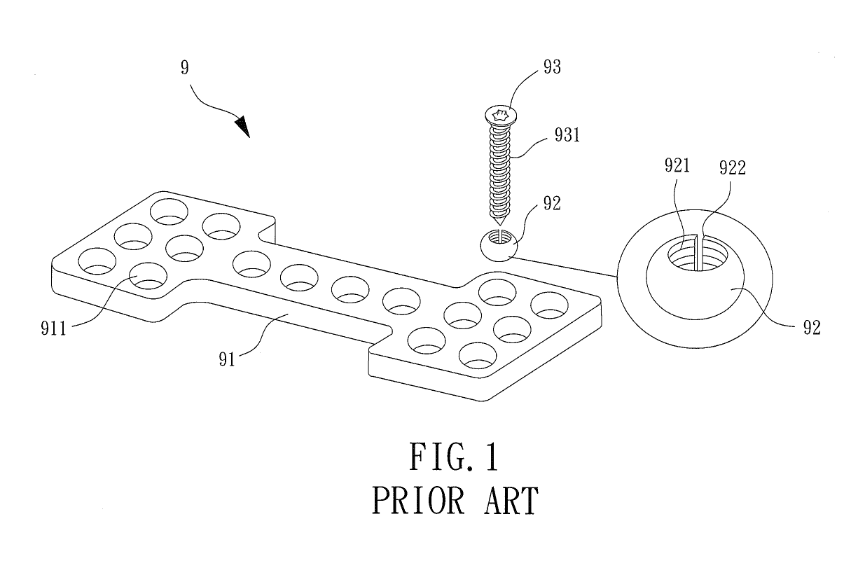

In the various figures of the drawings, the same numerals designate the same or similar parts. Furthermore, when the term “first”, “second”, “third”, “fourth”, “inner”, “outer” “top”, “bottom” and similar terms are used hereinafter, it should be understood that these terms refer only to the structure shown in the drawings as it would appear to a person viewing the drawings, and are utilized only to facilitate describing the invention.

### DETAILED DESCRIPTION OF THE INVENTION

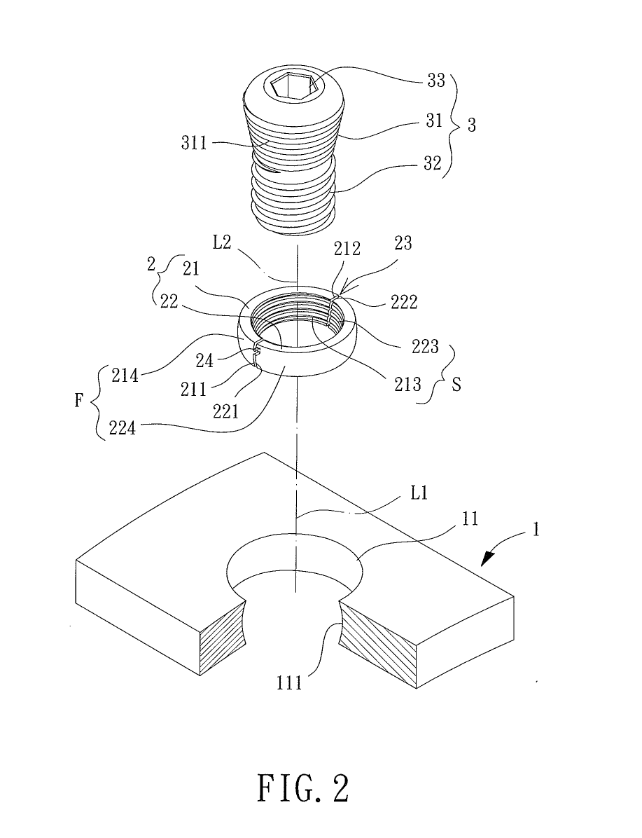

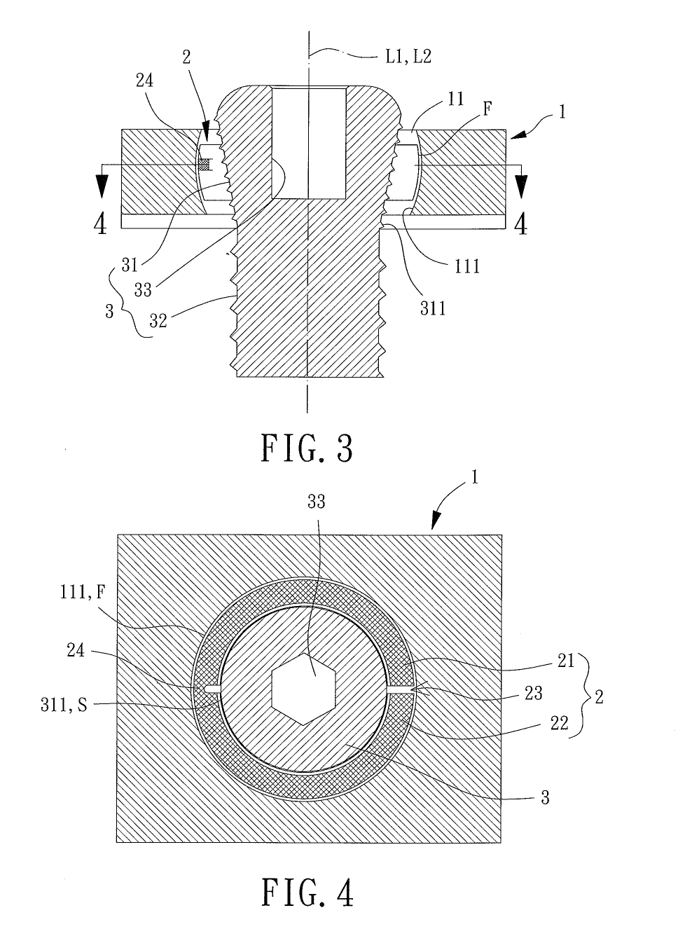

Referring to FIGS. 2 and 3, a bone plate module with adjustable fixing direction includes a bone plate 1, an adjustment ring 2 and a bone nail 3 according to a preferred embodiment of the invention. The bone nail 3 may fix the adjustment ring 2 to the bone plate 1.

The bone plate 1 may be implemented in various shapes fitted to an injured part with fracture, and has at least a coupling hole 11 for receiving the adjustment ring 2. The coupling hole 11 may have an arcuate circumferential wall 111 with a predetermined curvature that allows a user to fine adjust the position and angle of the adjustment ring 2 in the coupling hole 11. In other words, the arcuate circumferential wall 111 of the coupling hole 11 may allow the adjustment ring 2 to be disposed in the coupling hole 11 in different inclinations. In this embodiment, the arcuate circumferential wall 111 is a circumferential wall with outward arcuation in a radial direction. To elaborate how the adjustment ring 2 can be disposed in the coupling hole 11 in different inclinations, a first axial line L1 extending axially at a center of the coupling hole 11, as well as a second axial line L2 extending axially at a center of the adjustment ring 2, are herein defined.

When the adjustment ring 2 is disposed in the coupling hole 11 without any horizontal inclination (without deviating from the horizontal axis), the first axial line L1 will be overlapped with the second axial line L2 as shown in FIG. 3. Referring back to FIG. 2, the adjustment ring 2 comprises a first adjustment member 21 and a second adjustment member 22. Both the first adjustment member 21 and the second adjustment member 22 are of a semicircular form. The first adjustment member 21 has one end coupled with one end of the second adjustment member 22. The first adjustment member 21 has the other end that may be either coupled with the other end of the second adjustment member 22 or very close to but not coupled with the other end of the second adjustment member 22. In this embodiment, the first adjustment member 21 and the second adjustment member 22 are coupled at only one end while leaving the other end close but unconnected to each other.

Specifically, the first adjustment member 21 has a first end 211 and a second end 212, and the second adjustment member 22 has a first end 221 and a second end 222, with the first end 221 of the second adjustment member 22 and the first end 211 of the first adjustment member 21 connected together. Thus, the first adjustment member 21 and the second adjustment member 22 can be coupled with each other at the first ends 211 and 221 when a gap 23 is presented between the second ends 222 and 212. Note the first adjustment member 21 and the second adjustment member 22 are connected in a way that the first ends 211 and 221 are allowed to disengage from each other when the adjustment ring 2 experiences an outward radial expansion caused by an external force applied to hold open the adjustment ring 2. This outward radial expansion can increase the radial expansion range of the adjustment ring 2 until an outer circumferential face of the adjustment ring 2 tightly abuts against the arcuate circumferential wall 111 of the coupling hole 11. The first ends 211 and 221 can be coupled with each other by ways of, for example, adhesion or fastening. Alternatively, the first ends 211 and 221 can also be coupled with each other via a connection member 24 as shown in FIG. 2. Note the term “outward radial expansion” mentioned herein refers to an outward expansion that takes place in the radial direction perpendicular to the second axial line L2.

The adjustment ring 2 further comprises a thread portion S on an inner circumferential face thereof, as well as a coupling face F with a predetermined curvature on an outer circumferential face thereof. The thread portion S may be threadedly-engaged with the bone nail 3 until the bone nail 3 drives the adjustment ring 2 to an extent where the coupling face F of the adjustment ring 2 becomes tightly abutted against the arcuate circumferential wall 111 of the coupling hole 11. Specifically, the first adjustment member 21 may form a first thread portion 213 on an inner face thereof, as well as a first coupling face 214 on an outer face thereof. Similarly, the second adjustment member 22 may form a second thread portion 223 on an inner face thereof, as well as a second coupling face 224 on an outer face thereof. In such an arrangement, the first ends 211 and 221 should be connected in a way that the thread of the first thread portion 213 is aligned with that of the second thread portion 223, so as to form the thread portion S. Alignment of the threads between the first thread portion 213 and the second thread portion 223 can also provide alignment between the first coupling face 214 and the second coupling face 224, so as to form the coupling face F. Optionally, the unconnected second ends 212 and 222 of the first and second adjustment members 21 and 22 may also be connected together to ensure better alignments between the first thread portion 213 and the second thread portion 223, as well as between the first coupling face 214 and the second coupling face 224.

Specifically, referring to FIGS. 2 and 3, the thread portion S has a varying diameter that is increasingly reduced from top to bottom sides of the adjustment ring 2, and the coupling face F is implemented as an arcuate circumferential face having a curvature corresponding to the predetermined curvature of the arcuate circumferential wall 111. The match in curvature between the arcuate circumferential face (the coupling face F) and the arcuate circumferential wall 111 can allow the adjustment ring 2 to be disposed in the coupling hole 11 in different inclinations.

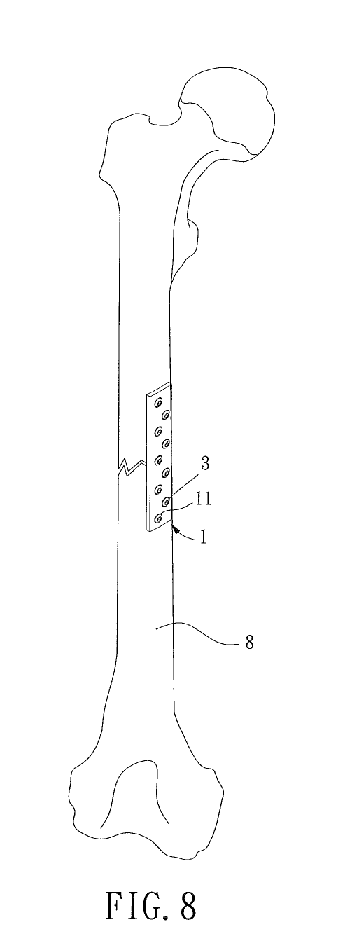

The bone nail 3 can be driven through the adjustment ring 2 to cause the outward radial expansion of the adjustment ring 2. This allows the adjustment ring 2 to be securely received in the coupling hole 11 of the bone plate 1. When the adjustment ring 2 is securely positioned in the coupling hole 11 of the bone plate 1, the bone plate 1 can be placed on the injured part, and the bone nail 3 can be driven through the adjustment ring 2 to fix into a bone 8 of the injured part, as shown in FIG. 8. Specifically, the bone nail 3 has an adjustment portion 31 and a screwing portion 32. The adjustment portion 31 can force the adjustment ring 2 to expand outwards radially, and the screwing portion 32 can be screwed into the bone 8. In this embodiment, the adjustment portion 31 has a varying diameter that is increasingly reduced from upper to lower portions of the adjustment portion 31. Further, the adjustment portion 31 has a thread portion 311 that can be threadedly-engaged with the thread portion S of the adjustment ring 2.

The bone nail 3 can further include a driving hole 33 adjacent to the adjustment portion 31 on one end thereof. A proper tool may be inserted into the driving hole 33 to drive the bone nail 3 through the adjustment ring 2 and into the bone 8 of the injured part.

Referring to FIGS. 3, 4 and 8, when in use, the adjustment ring 2 is disposed into the coupling hole 11 of the bone plate 1. Then, the bone plate 1 is placed on the bone 8 of the injured part. Finally, the screwing portion 32 of the bone nail 3 is driven through the adjustment ring 2 until the adjustment portion 31 of the bone nail 3 is aligned with the thread portion S of the adjustment ring 2.

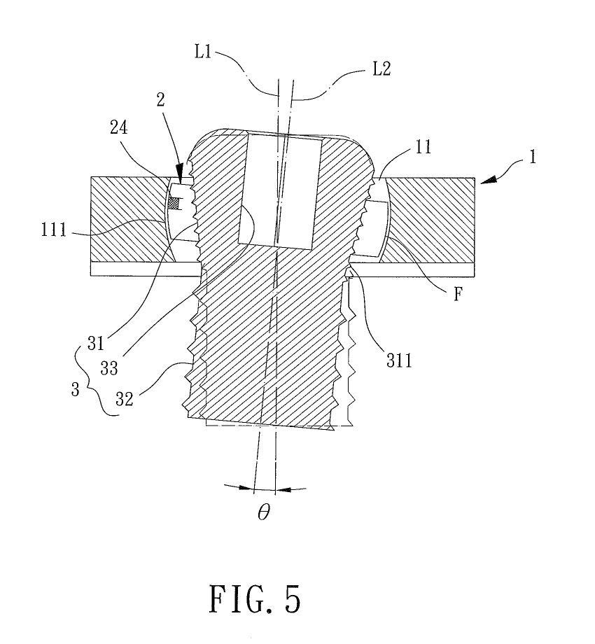

Referring to FIG. 5, since the arcuate circumferential wall 111 of the coupling hole 11 and the coupling face F of the adjustment ring 2 correspond to each other (having the same curvature), the adjustment ring 2 can be disposed in the coupling hole 11 in different inclinations. At this point, an included angle θ will be formed between the first axial line L1 and the second axial line L2. Thus, the adjustment ring 2 can be disposed in the coupling hole 11 in a desired inclination that allows the bone nail 3 to be extended through the adjustment ring 2 in a certain angle where the screwing portion 32 of the bone nail 3 can be best screwed into the bone. This allows the bone nail 3 to be fixed into the bone in multiple directions and positions.

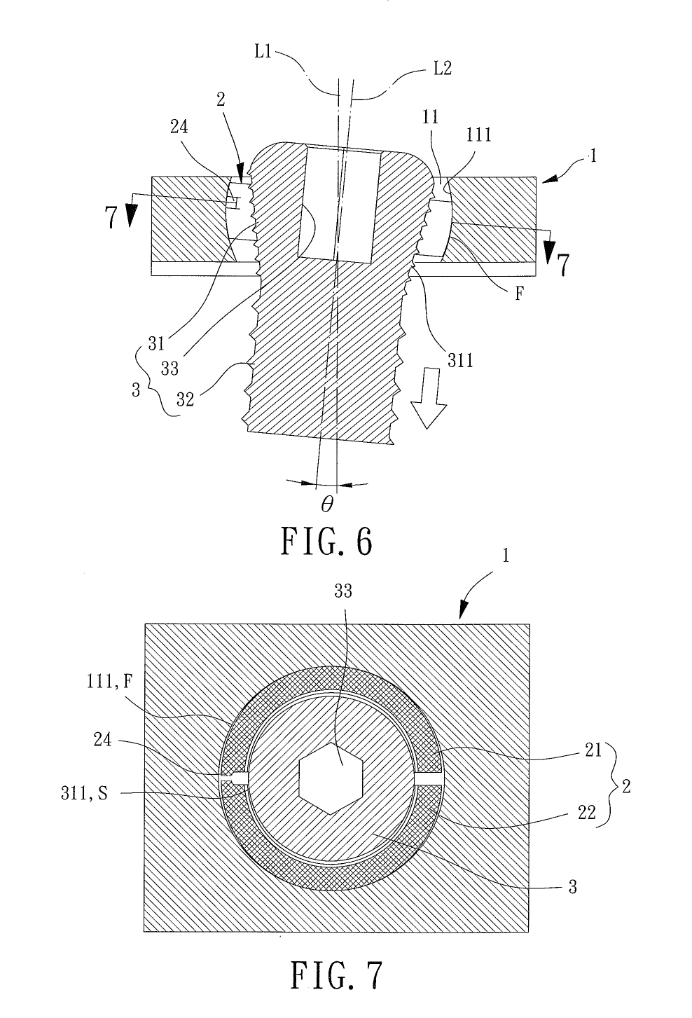

Referring to FIGS. 6, 7 and 8, since the adjustment portion 31 of the bone nail 3 and the thread portion S of the adjustment ring 2 correspond to each other (having the same varying diameter), the adjustment portion 31 can force the adjustment ring 2 to expand outwards radially when the screwing portion 32 is being screwed into the bone. The adjustment portion 31 will force the adjustment ring 2 to keep expanding until the coupling face F of the adjustment ring 2 touches and abuts against the arcuate circumferential wall 111 of the coupling hole 11. Note the coupling face F of the adjustment ring 2 and the arcuate circumferential wall 111 of the coupling hole 11 can both be rough to increase the surface friction therebetween. The roughness prevents the adjustment ring 2 from rotating in the coupling hole 11 when the bone nail 3 is driven into the bone, thereby enhancing the coupling effect between the bone nail 3 and the bone plate 1. More importantly, the adjustment portion 31 can force the first ends 211 and 221 to disengage from each other when the screwing portion 32 of the bone nail 3 is driven into the bone by a predetermined depth. This can increase the radial expansion range of the adjustment ring 2. In other words, the connection member 24 will break when the adjustment ring 2 has expanded to a certain extent, leaving the first adjustment member 21 and the second adjustment member 22 unconnected as shown in FIG. 7. At this moment, the adjustment portion 31 can eventually drive the first adjustment member 21 and the second adjustment member 22 away from each other when the bone nail 3 is driven further into the bone. This allows the first coupling face 214 and the second coupling face 224 to securely abut against the arcuate circumferential wall 111 of the coupling hole 11, so that the bone plate 1 can be stably positioned on the bone 8. Thus, healing of the injured part with fracture can be facilitated.

The bone plate module of the invention is characterized in connecting the first adjustment member 21 and the second adjustment member 22 together via the first ends 211 and 221, so that the adjustment portion 31 of the bone nail 3 can be threadedly-engaged with the thread portion S. When the adjustment portion 31 is threadedly-engaged with the thread portion S, the bone nail 3 can force the adjustment ring 2 to expand outwards radially and finally cause the disengagement between the first adjustment member 21 and the second adjustment member 22. In this way, the radial expansion range of the adjustment ring 2 can be significantly increased, allowing the first adjustment member 21 and the second adjustment member 22 to be securely positioned in the coupling hole 11. This can stably position the bone plate 1 on the bone 8 for improved healing effect of the injured part.

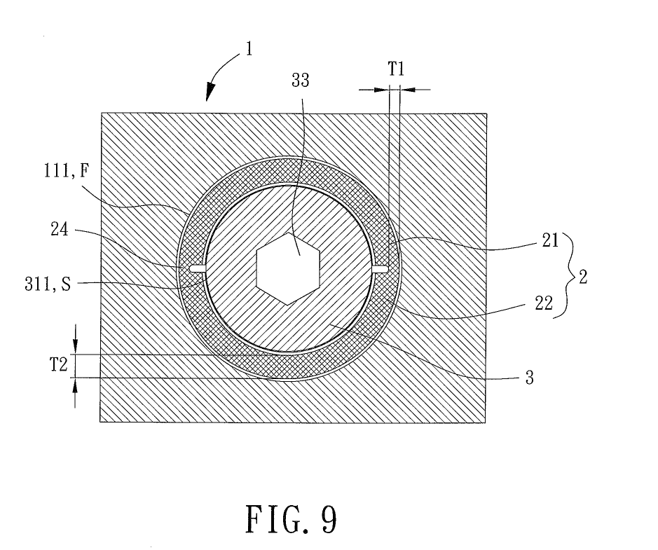

As described above, the second ends 212 and 222 of the first and second adjustment members 21 and 22 can also be coupled with each other via another connection member 24 or by ways of, for example, adhesion or fastening. As shown in FIG. 9, the second ends 212 and 222 of the first and second adjustment members 21 and 22 are coupled together via the connection member 24. Note the connection member 24 has a radial thickness T1 smaller than a radial thickness T2 of the adjustment ring 2. Thus, the connection members 24 can easily break when the adjustment ring 2 has expanded to a certain extent due to the bone nail 3 driven therethrough.

Although the invention has been described in detail with reference to its presently preferable embodiment, it will be understood by one of ordinary skill in the art that various modifications can be made without departing from the spirit and the scope of the invention, as set forth in the appended claims.

What is claimed is:

1. A bone plate module with adjustable fixing direction, comprising:
a bone plate having a coupling hole;
an adjustment ring disposed in the coupling hole and having a thread portion, wherein the adjustment ring further comprises a first adjustment member and a second adjustment member, and the first adjustment member has a first end coupled with a first end of the second adjustment member; and
a bone nail having an adjustment portion coupled with the thread portion of the adjustment ring.

2. The bone plate module with adjustable fixing direction as claimed in claim 1, wherein the first ends of the first and second adjustment members are connected via a connection member.

3. The bone plate module with adjustable fixing direction as claimed in claim 1, wherein the first adjustment member further comprises a second end coupled with a second end of the second adjustment member.

4. The bone plate module with adjustable fixing direction as claimed in claim 1, wherein the coupling hole has a circumferential wall with outward arcuation in a radial direction, the adjustment ring has a coupling face in the form of an arcuate circumferential face corresponding to the circumferential wall of the coupling hole, and both the circumferential wall of the coupling hole and the coupling face of the adjustment ring are rough.

5. The bone plate module with adjustable fixing direction as claimed in claim 1, wherein both the thread portion of the adjustment ring and the adjustment portion of the bone nail have a varying diameter that is increasingly reduced from one side to the other side thereof.

6. An adjustment ring of a bone plate module with adjustable fixing direction, comprising:
a first adjustment member having a first end and a first thread portion; and
a second adjustment member having a first end and a second thread portion, wherein the first ends of the first and second adjustment members are connected to each other, and the first and second thread portions jointly form a thread portion.

7. The adjustment ring of the bone plate module with adjustable fixing direction as claimed in claim 6, wherein the first ends of the first and second adjustment members are connected via a connection member.

8. The adjustment ring of the bone plate module with adjustable fixing direction as claimed in claim 6, wherein the adjustment ring has a coupling face on an outer circumferential face thereof, and the coupling face is rough and in the form of an arcuate circumferential face.

9. The adjustment ring of the bone plate module with adjustable fixing direction as claimed in claim 6, wherein the thread portion has a varying diameter that is increasingly reduced from one side to the other side thereof.

10. The adjustment ring of the bone plate module with adjustable fixing direction as claimed in claim 6, wherein the first adjustment member further comprises a second end coupled with a second end of the second adjustment member.

11. The adjustment ring of the bone plate module with adjustable fixing direction as claimed in claim 7, wherein the connection member has a radial thickness smaller than that of the adjustment ring.
